(19) United States
(12) Patent Application Publication
(10) Pub. No.: US 2015/0273454 A1
(43) Pub. Date: Oct. 1, 2015

(54) CATALYST FOR CELLULOSE HYDROLYSIS

(71) Applicant: Stephen ROTH, Gladwyne, PA (US)

(72) Inventor: Stephen ROTH, Gladwyne, PA (US)

(73) Assignee: United Catalyst, LLC, Gladwyne, PA (US)

(21) Appl. No.: 14/502,146

(22) Filed: Sep. 30, 2014

Related U.S. Application Data
(60) Provisional application No. 61/972,853, filed on Mar. 31, 2014.

(51) Int. Cl. B01J 31/06; C07C 29/132; B01J 37/02; C07H 3/02; B01J 35/04; B01J 37/06; C07H 1/06; C07H 1/08

(52) U.S. Cl. CPC B01J 31/069; C07H 1/06; C07C 29/132; C07H 1/08; C07H 3/02; B01J 35/04; B01J 37/06; B01J 37/0211; B01J 2231/005; B01J 2531/004; B01J 2531/008

(57) ABSTRACT

The present invention is directed to an imprinted mesoporous silica catalyst functionalized with a carboxylic acid group, capable of binding and hydrolyzing at least one glucose substrate into glucose.

### BACKGROUND OF THE INVENTION

1. Field of the Invention

The present invention is directed to an imprinted mesoporous silica catalyst functionalized with a carboxylic acid group, capable of hydrolyzing at least one glucose substrate into glucose.

2. Discussion of the Background

Refined petroleum products have been a major energy source for many decades. Unfortunately, the prices for both refined and crude petroleum-based products have risen dramatically in recent years due to increased global demand and finite supplies. Moreover, petroleum products, when burned, contribute substantially to the release of CO₂ into the atmosphere. As a result of the many issues associated with petroleum products, there is substantial interest in exploring alternative energy sources. One alternative to petroleum-based fuel is ethanol.

Ethanol is typically produced by yeast fermentation of either sucrose or glucose. The viability of replacing petroleum-based fuels with ethanol, on a regional scale, has already been successfully demonstrated. In Brazil, for example, sugar cane-derived ethanol has largely replaced petroleum-based fuels. (Sperling, D.; Gordon, D., Two billion cars: driving toward sustainability. Oxford University Press: New York, 2009).

Although ethanol has shown promise as an alternative fuel source, there are many obstacles that have hampered the growth of ethanol production in the United States. Specifically, the raw materials necessary to produce ethanol, e.g., sucrose and glucose, are common food stuffs. Sucrose, for example, is a non-reducing disaccharide consisting of glucose and fructose, and is produced primarily from cane sugar or sugar beets. Glucose, produced by the hydrolysis of plant starches (repeating polymers of α-linked glucosides), is derived predominantly from corn. The demand for the raw materials necessary to produce ethanol competes with the demand for food, resulting in increased prices for both food and fuel. The potential benefits of ethanol derived from corn are further diminished by studies that have shown that corn-based ethanol could have a net climate warming effect. (Crutzen, P. J., et al., Atmospheric Chemistry and Physics 2008, 8, (2), 389-395.)

In an attempt to mitigate some of the issues related to the production of corn-based ethanol, the ethanol industry has turned its attention to methods of developing glucose from cellulose. Cellulose is a polymer consisting of β-1,4-linked glucosides and can be found in nearly all plant materials. Thus, an efficient method of hydrolyzing cellulose into glucose could allow ethanol production facilities to access the estimated 10¹¹ tons per year of cellulose normally produced by plants on earth.

Large amount of cellulose could even be obtained from crop remains such as corn stover, cane bagasse, and even cellulose-based trash. These materials would be sustainable, easy to collect, and very inexpensive (Saha, B. C., et al. In Fuel ethanol production from corn fiber-Current status and technical prospects, 1998; Humana Press Inc: 1998; pp 115-125; Tucker, M. P., et al. In Conversion of distiller's grain into fuel alcohol and a higher-value animal feed by dilute-acid pretreatment, 2004; Humana Press Inc: 2004; pp 1139-1159). Another advantage of cellulose over cane sugar is that cellulose hydrolysis will yield only glucose, which yeast favor over sucrose.

Even though cellulose is easily the most abundant biological material on earth, it is not trivial to hydrolyze cellulose directly to glucose. Given the difficulties associated with the hydrolysis, most efforts at large-scale cellulose hydrolysis have been focused on using cellulases, a class of enzymes that catalyze the hydrolysis of cellulose. Research has also focused on the preparation of modified enzymes engineered to be more stable under the extreme high temperatures and pH conditions required to hydrolyze cellulose. (Sun, Y. et al. Bioresource Technology 2002, 83, (1), 1-11; Wright, J. D. Chemical Engineering Progress 1988, 84, (8), 62-74.)

Despite some success in preparing cellulases capable of hydrolyzing cellulose to glucose, even the most heat-stable cellulases are expensive and relatively short-lived. Moreover, enzyme-based hydrolysis typically requires several days to achieve desirable reactions. It is also difficult to separate and reuse cellulase enzymes, making any process using these reagents expensive. The combination of these various issues impedes the economical production of ethanol from lignocellulosic material. It would therefore be useful to develop and use one or more inorganic catalysts that can mimic an exoglucosidase (an enzyme that cleaves a terminal glucose residue from a cellulose oligo- or polysaccharide) and/or an endoglucosidase (an enzyme that cleaves the glucose polymer at an internal linkage).

Inorganic catalysts, unlike their biological counterparts, can successfully tolerate harsh conditions and can be used repeatedly without loss of activity. Moreover, even if an inorganic catalyst were less active then currently known enzymes, the inorganic catalyst could have significant commercial importance. For example, a silica-based catalyst 100 times less active than a corresponding enzyme, but 1,000 times less expensive to produce, and 100 times more stable under the conditions necessary for cellulose hydrolysis, would be a commercially attractive alternative to enzyme based technology.

Recent efforts to prepare inorganic catalysts for various purposes have focused on a strategy employing molecular imprinting. (Gupta, R., et al. Biotechnology Advances 2008, 26, (6), 533-547; Katz, A., et al. Nature 2000, 403, (6767), 286-289.) In molecular imprinting, an imprinting template acts as a form around which cross-linkable monomers are co-polymerized to form a cast-like shell. Without wishing to be bound by any particular theory, it is believed that the monomers in a given imprinting reaction form a complex with the template through covalent and/or non-covalent interactions. The monomers are subsequently polymerized in the presence of the template.

After polymerization, the imprinting template is removed, exposing cavities that are complementary to the template in size and shape. These cavities, essentially negative images of the imprinting templates, are subsequently capable of selectively rebinding the templates, or molecules similar to the templates. The template-free polymer or copolymer can be referred to as a “molecularly imprinted polymer” (“MIP”). MIPs possess important features of biological receptors-recognition.

Roth et al. U.S. 2012/0136180 describes an imprinted biomimetic catalyst for cellulose hydrolysis.

### SUMMARY OF THE INVENTION

The present invention is directed to an imprinted mesoporous silica catalyst functionalized with a carboxylic acid group capable of binding and hydrolyzing at least one glucose substrate to glucose. Applicants have discovered an imprinted catalyst functionalized with a carboxylic acid group to display a higher activity than previously demonstrated imprinted catalysts.

The present invention further includes a method for preparing a biomimetic catalyst. The method includes the steps of reacting at least one tetraorthosilicate with at least one functionalized silane in the presence of an imprinting molecule to form a polymeric silica matrix impregnated with said imprinting molecule. At least 1 mol percent of said at least one silane is a carboxylic acid or carboxylic acid precursor functionalized silane. The method further includes isolating the impregnated polymeric silica matrix; and removing said imprinting molecule via washing or burning to form a silica matrix imprinted with the structure of said imprinting molecule.

The present invention further includes a method for preparing a biomimetic catalyst, said method comprising reacting at least one tetraorthosilicate and at least one functionalized silane, in the presence of at least one functionalized imprinting molecule, to form a polymeric silica matrix impregnated with said functionalized imprinting molecule; isolating said polymeric silica matrix so impregnated; and removing said imprinting molecule from said polymeric silica matrix so impregnated via hydrolysis or pyrolysis to yield a silica matrix imprinted with the structure of said imprinting molecule.

In light of the utility of Si-based polymers, the catalyst described herein is a polymeric silica matrix that has been imprinted with one or more imprinting molecules, resulting in a catalyst including at least one, and in certain embodiments, a plurality of active sites. The active sites of the catalyst are capable of binding one or more varieties of glucose substrate. Once the active site binds a glucose substrate, active functionalities located within the active site react with the glucose substrate to break or otherwise assist in the breaking of glycosidic bonds.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 illustrates the hydrolysis of a β-glucan at 150° C. as a function of an Avicel-imprinted catalyst bearing a sulfonic acid group (AVI-MIP) or a carboxylic acid group (AVI-MIP-CA);

FIG. 2 illustrates the hydrolysis of a β-glucan at 150° C. as a function of time with an imprinted catalyst bearing a sulfonic acid group (AVI-MIP); and

FIG. 3 illustrates the hydrolysis of a β-glucan at 150° C. as a function of the imprinted catalyst after 47 hours.

### DETAILED DESCRIPTION OF THE PREFERRED EMBODIMENTS

### Silane Based Biomimetic Catalyst

The silica matrix of the catalyst can be prepared using sol/gel chemistry. One major advantage of molecularly imprinted Si-based sol-gel networks over natural or engineered enzymes is superior thermal stability. Silicon based materials can easily withstand the high temperatures and low pHs used for hydrolysis of cellulose on an industrial scale. Additional benefits of the Si-based catalysts over naturally occurring enzymes include recoverability and cost effectiveness.

At least some of the silane is functionalized with a carboxylic acid group or carboxylic acid precursor, so that the catalyst produced according to the present method will be able to cleave glycosidic linkages. Applicants have discovered that an imprinted silica functionalized with a carboxylic acid group to provide for a higher catalytic activity in the cleavage of β-1,4-linked glucosides, that the corresponding imprinted catalyst functionalized with a sulfonic acid group.

Particularly useful cross-linked polymers that are susceptible to imprinting are Si-based polymers. These polymers include many Si—O linkages and a smaller percentage of Si—R linkages, wherein R can be variously substituted alkyl, aryl, or other non-oxygen functionality.

The at least one tetraorthosilicate is selected from the group consisting of Formulas I, II, III, and IV:

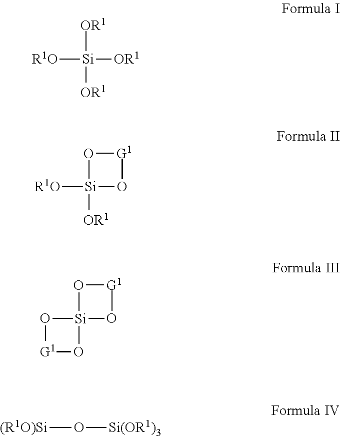

wherein R¹ is, independently at each occurrence, C₁₋₆ alkyl or phenyl;

G¹ is independently C₁₋₆ alkyl or a bivalent cyclic group selected from the group consisting of

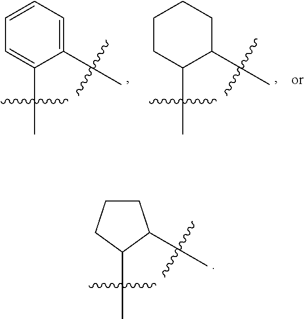 [SMILES: *c1ccccc1* ; *C1CCCCC1* ; *C1CCCC1*]

Functionalized silanes useful for preparing the silica matrix of the catalyst described herein can have structures according to any of Formulas V, VI, or VII.

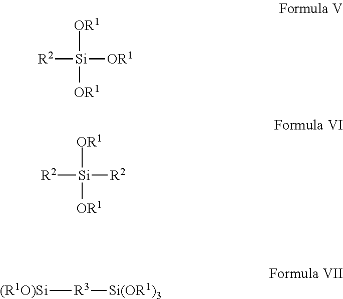

In the formulas above, R¹ can be, independently at each occurrence, C₁₋₆ alkyl or phenyl.

R² is independently at each occurrence, C₁₋₆ alkyl optionally substituted with at least one substituent selected from the group consisting R⁴ and

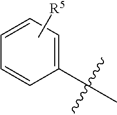

provided that at least one group R² is substituted with a carboxylic acid or a nitrile group.

Alternatively, R² can be

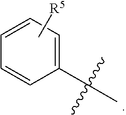

R³ can be C₁₋₆ alkyl optionally substituted with a substituent selected from the group consisting of R⁴ and

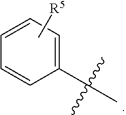

R⁴ can be CN, SH, NH₂, OH, CO₂H, or SO₃H.

R⁵ can be CN, H, SO₂CH₃, NH₂, OH, CO₂H, or SO₃H.

G¹ can be C₁₋₆ alkyl (such that an optionally C₁₋₅ alkyl substituted 1,3,2-dioxasiletane, an optionally C₁₋₄ alkyl substituted 1,3,2-dioxasilolane, or an optionally C₁₋₃ alkyl substituted 1,3,2-dioxasilinane is formed),

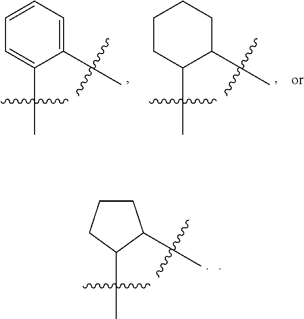 [SMILES: *c1ccccc1* ; *C1CCCCC1* ; *C1CCCC1*]

An imprinted silica catalyst having carboxylic acid functionalities may be obtained by reacting a carboxylic acid precursor-containing silane of formula V-VII, wherein R⁴ is a nitrile group. After formation of the imprinted silica catalyst bearing a nitrile group, an imprinted silica catalyst bearing a carboxylic acid group may be obtained by hydrolysis of the nitrile group under conditions known to those of ordinary skill in the art, such as under the action of an inorganic acid, such as sulfuric acid, hydrochloric acid, nitric acid and hydrofluoric acid at a temperature of from 50-120° C., preferably ˜98° C.

Examples of additional acid functionalized silanes include, but are not limited to, those compounds of Formulas V, VI, and VII described previously, as well as compounds such as triethoxysilylpropylmaleamic acid, and N-(trimethoxysilylpropyl)ethylenediamine, triacetic acid, trisodium salt.

In certain embodiments, the quantity of carboxylic acid functionalized silane can be from about 1 to about 50 mol percent of the total quantity of silane used to prepare the polymeric material, inclusive of any and all whole or partial increments therebetween, with the remainder being unfunctionalized silane. In specific embodiments, the quantity of carboxylic acid functionalized silane can be from about 1 to about 40 percent, from about 1 to about 30 percent, from about 1 to about 25 percent, from about 1 to about 20 percent, from about 1 to about 15 percent, from about 1 to about 10 percent, or from about 1 to about 5 mol percent of total quantity of silane.

Alternatively the content of silane may be in a ratio of 1:1-100, preferably 1:5-20, more preferably 1:10 based on tetraalkyl orthosilicate.

When either R⁴ is SH or R⁵ is SO₂Cl, the thiol or chlorosulfonyl groups, can be oxidized to SO₃H in order to provide the functional groups appropriate for hydrolyzing a given substrate. When R⁴ is SH, the oxidant can be, for example, H₂O₂. When R⁵ is SO₂Cl, the oxidant can be, for example, water.

The polymerization of the tetraorthosilicate and the silane(s) can be acid or base catalyzed and can takes place in a variety of solvents, including water, standard organic solvents, and ionic liquids. Examples of suitable ionic liquids include, but are not limited to N-ethylpyridinium chloride, BMIM⁺X⁻ (BMIM⁺1-butyl-3-methylimidazolium) wherein X=.Cl, Br, SCN, BF₄, or PF₆, and AMIM⁺Cl⁻ (AMIM=1-allyl-3-methylimidazolium chloride). Useful organic solvents include, but are not limited to, N,N-DMF, methanol, ethanol, isopropanol, ethyl acetate, isopropyl acetate, dichloromethane, chloroform, NMP, tetrahydrofuran (THF), acetonitrile, as well as mixtures thereof.

Suitable acidic catalysts include, but are not limited to, anhydrous and aqueous HCl, p-toluenesulfonic acid, sulfuric acid, benzene sulfonic acid, camphor sulfonic acid, and other acids known to those of ordinary skill in the art. Suitable basic catalysts include NaOH, CaOH₂, MgOH₂, and NH₄OH.

Molecular imprinting with an imprinting molecule takes place during the polymerization reaction. In one embodiment of the imprinting process, the imprinting molecule is added directly to the polymerization reaction. The quantity of imprinting molecule added will be dependent on several factors, including the size (molecular weight) and solubility of the imprinting molecule. Anywhere from about 1 to about 99 mol percent of the imprinting molecule including any whole or partial increment therebetween (based on the total molar quantity of silane and tetraorthosilicate) can be added.

Silica preparation and imprinting may be conducted under sol-gel conditions known to those of ordinary skill in the art. A reaction temperature from 20-150° C., preferably 40-120° C., more preferably 50-90° C. may be used.

For each of the embodiments described above, the polymeric matrix forms around imprinting molecules present during the polymerization reaction. As a result, the imprinting molecules become impregnated in or on the silica matrix.

Once the polymerization is complete, the solvent in the reaction flask can be removed. Solvent removal can take place before or after a first milling step, depending upon the solubility of the catalyst matrix. When the resulting polymeric matrix is soluble in the reaction solvent, the solvent can be removed under reduced pressure using, for example, a rotary evaporator. Alternatively, solvent can be removed using one of variously known freeze drying techniques. In another embodiment, solvent can be washed away with supercritical CO₂. When the resulting silica matrix is not soluble in, or is substantially less soluble in the reaction solvent than the starting materials, the silica matrix material can be filtered away from the reaction solvent.

Although standard evaporative techniques can be used to remove solvent from the reaction vessel, it is not a preferred methodology. Specifically, removing solvent under reduced pressure creates surface tension at or near the newly formed active sites in the silica matrix. This surface tension can damage, shrink, or otherwise deform the matrix during the solvents' liquid/gas transition. Thus, a process which mitigates these potential pitfalls is preferable. One such method is critical point drying.

Critical point drying using supercritical CO₂ is useful as it mitigates any issues associated with surface tension resulting from a typical volatilization processes. The supercritical CO₂ wash can also act to burn off any imprinting molecules present in the matrix if performed at high enough temperature.

The dry solid can then be milled to give silica particles having specific sizes. Upon completion of the milling process, particles can be washed with solvent to remove any imprinting molecules revealed by the process. The particles can then be dried with supercritical CO₂. Alternatively, or additionally, the milled solids can be heated to a sufficiently high temperature so that any imprinting molecules present in the milled solids are burned away. Once all of the imprinting molecules have been removed, the resultant silica particles can be tested for efficacy.

### Imprinting Molecule

The imprinted silica catalyst having carboxylic acid functionalities is formed in the presence of an imprinting molecule which provides a negative image of the imprinting molecule in the catalyst. Suitable imprinting molecules are those bearing glucan linkages of the β-1,4 type as well as of the β-1,3 and β-1,6 types. Non-limiting examples of suitable imprinting molecules are cellobiose (two glucose molecules with a β-1,4 linkage), hydroxyethyl cellulose, Avicel and cellulose (polyglucoses with β-1,4 linkages) and beta-glucan (a polysaccharide of D-glucose monomers linked by 1,3 and 1,6 β-glycosidic bonds). It is within the scope of the present invention to form imprinted mesoporous silica catalysts in the presence of one or more different imprinting molecules providing an imprinted silica having negative images of different glucan linkages.

In another embodiment of a method for preparing the catalyst described herein, a functionalized imprinting molecule can be employed. A functionalized imprinting molecule is an imprinting molecule that has been impermanently bound to a poly-functional linker through, for example, an ester linkage, sulfonic acid ester linkage, a carbamate (urethane), or a carbonate linkage.

Examples of poly-functional linkers include, but are not limited to, compounds of Formulas VIII-XI. In each of these compounds, R¹ is as described herein previously. G² is optional such that when G² is absent, a single bond is formed between the carbon and silicon shown linked to G². When present, G² is C₁₋₆ alkyl. G³ can be O or N.

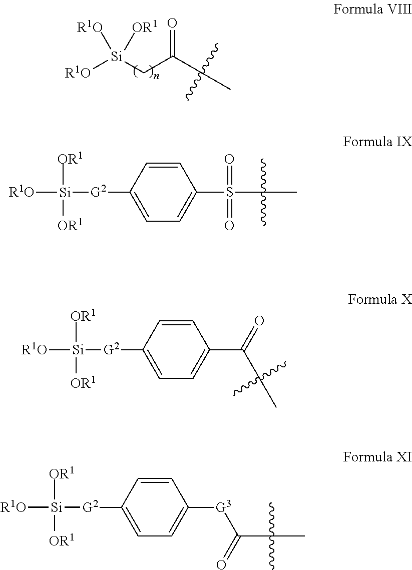

Although the silicon species in each of the compounds according to Formulas IX-XI are shown as para substituted, compounds of Formulas IX-XI can be functionalized with copies of the para Si functionality at one or both meta positions instead. Alternatively, the phenyl ring can be para Si substituted as shown, with the phenyl ring further functionalized with the Si group present at the para position at one or both ortho positions, provided sterics permit.

The poly-functionalized linkers can be used in the synthesis of a biomimetic catalyst according to the following procedure. First, a poly-functionalized linker precursor, such as an activated ester, acid chloride, sulfonyl chloride (e.g. 2-(4-chlorosulfonylphenyl)ethyltrimethoxysilane), isocyanate, or other appropriately reactive species is reacted with a free alcohol on an imprinting molecule, such as, for example cellobiose. The resulting product is a functionalized imprinting molecule. The number of linkers that can be attached to a given imprinting molecule will be determined by the reactivity of the linker precursor, the nucleophilicity of free alcohol(s) on the imprinting molecule, and steric interactions in and around the nucleophilic alcohol(s).

The functionalized imprinting molecule is then mixed with at least one tetraorthosilicate according to at least one of Formulas I, II, III or IV and, optionally, at least one silane according to at least one of Formulas V, VI, or VII. The reagents are then polymerized using an acidic catalyst. Solvents and catalysts useful for this polymerization have been identified herein previously. The molar ratios of the various components can range from about 100:20:80 to about 100:95:5, including all whole or partial increments there between. Ratios are presented as molar amounts of tetraorthosilicate:silane:functionalized imprinting molecule.

Once the polymerization is complete, the silica matrix can be isolated and milled according to the procedures set forth elsewhere herein. Subsequently, the imprinting molecule must be excised from the matrix. This can be accomplished via hydrolysis of the various carbonate, carbamate (urethane), carboxylic, and sulfonate esters binding the imprinting molecule to the silica matrix. Hydrolysis can be accomplished by using strongly acidic or basic conditions. The silica matrix can then be isolated from the imprinting molecule using any of the drying/burning techniques discussed herein previously. Subsequent milling is optional. The activity of the optionally milled catalyst can be assayed using known techniques.

When a carbamate (urethane) linker is utilized, the resulting polymer (after hydrolysis) will contain a substituted aniline in the active site. In certain embodiments, the aniline can be allowed to remain. In other embodiments, however, the amine of the aniline can be diazotized using a known reagent, such as nitrosonium tetrafluoroborate (NOBF₄) or sodium nitrite/H₂SO₄, and converted to a phenol, nitrile, or halide. If converted to a nitrile, the nitrile can subsequently be hydrolyzed to a carboxylic acid. If converted to a halide, the compound can be converted into a Grignard reagent and subsequently quenched with CO₂ to form a carboxylic acid. Alternatively, the halide can be cross coupled with another reagent using known Pd or Pt mediated cross coupling reactions.

The benefits of using the poly-functionalized linker, as compared to the non-linked procedures (described elsewhere herein) are manifest. Specifically, when the functionalized imprinting molecule is polymerized with the tetraorthosilicate and silane, the acidic residues that will subsequently be used to hydrolyze glucose substrates are positioned in the cavity created by the imprinting molecule in such a way as to make it more likely that a glucose substrate will be hydrolyzed to glucose.

### Hydrolysis of a Glucose Substrate

Having prepared a catalyst as described above, the catalyst can be used according to any of the following procedures. In a first embodiment, the catalyst and glucose substrate can be dissolved in a solvent. The imprinted mesoporous silica catalyst may be imprinted with a single imprinting molecule or imprinted with a plurality of imprinting molecules. In addition, the catalyst may be a mixture of imprinted mesoporous silica catalysts, each imprinted with a different imprinting molecule.

Appropriate solvents include, for example, ionic liquids, examples of which were described previously herein. Ionic liquids, unlike many organic solvents are capable of solvating glucose substrates such as cellulose and its derivatives. Although ionic liquids are useful, standard organic solvents such as water, N,N-DMF, methanol, ethanol, isopropanol, ethyl acetate, isopropyl acetate, dichloromethane, chloroform, carbon tetrachloride, NMP, tetrahydrofuran (THF), acetonitrile, as well as mixtures thereof, can also be used. The utility of any of the above described solvents will, however, depend on how soluble (or insoluble) the glucose substrate is in the solvent as well as the temperature at which the reaction is run.

In certain embodiments, the hydrolysis reaction can be performed using an ionic liquid in combination with another solvent, such as water, methanol, ethanol, isopropanol, or other higher alcohol. In particular embodiments, the solvent combination is an ionic liquid and water. Without wishing to be bound to any particular theory, it is believed that when an ionic liquid and water are used as the solvents, and the amount of ionic liquid used is smaller than the amount of water, the catalyst will sequester the ionic liquids in micelle like structures, creating an emulsion wherein the micelle-like structures are microreactors for the hydrolysis of a glucose substrate. It is believed, that the catalyst membrane will be semi-permeable such that glucose will be able to exit the microreactor after glucose substrate hydrolysis. These microreactors will enable specific binding of individual cellulose polymers (or other glucose substrate) to catalytic domains present on and/or in the catalyst and, at the same time, allow for the subsequent release of glucose based on glucose's high solubility in the aqueous phase. The catalysts can be readily recycled through emulsion destabilization and re-emulsification process to achieve a high rate of cellulose conversion.

In alternative embodiments, the ionic liquid can be used in an amount (volume) in excess of the quantity of water used. Without wishing to be bound to any particular theory, it is believed that in this configuration, water will be sequestered by the catalyst to form micelle like structures. Catalysts would take place at the ionic liquid/catalyst interface and the glucose thus produced would migrate into the aqueous layer.

Suitable glucose substrates may be any glucan containing substrate such as, but not limited to corn stover, cane bagasse, sawdust, timber, forest waste, starches, amylose, fruit rinds, agriculture waste and celluose-based trash. Another suitable substrate may be waste paper pulp and paper pulp fines produced in a paper making process.

The imprinted mesoporous silica may also be used to increase the nutritional content of cellulose containing animal feeds. The imprinted mesoporous silica may be used to degrade a cellulosic substrate and used as a component in animal feed. Alternatively the imprinted mesoporous silica may be added to an animal feed containing a cellulosic substrate, enhancing decomposition of a cellulosic substrate in vivo.

Generally the glucose substrate contains β-1,4-linked glucosides. In one embodiment, the glucose substrate is a substrate containing up to 1,500 saccharide units, preferably up to 1,200 saccharide units, more preferably up to 1,000 saccharide units. Preferably the glucose substrate contains at least 10 saccharide units, more preferably at least 100 saccharide units, even more preferably at least 500 saccharide units.

The preparation of a glucose substrate contains β-1,4-linked glucosides and containing up to 1,500 saccharide units is within the level of skill of those of ordinary skill in the art.

The hydrolysis reaction can be run at temperatures ranging from about 40° C. to about 400° C., including all whole and partial increments there between. In one embodiment, the hydrolysis reaction is run at about 100 to about 150° C. The temperature at which the reaction will be run will dictate which solvents can be used. Catalyst loading in a given reaction can be from 0.01 weight percent to 50 weight percent based on the weight of glucose substrate present in the reaction. In preferred embodiments, the catalyst loading is less than about 10 weight percent, even more preferably less than about 5 weight percent and most preferably less than about 1 weight percent.

The imprinted mesoporous silica catalyst functionalized with a carboxylic acid group has high catalytic activity in the cleavage of glucosides from a glucose substrate. The catalytic activity is preferably at least 20 μmoles·min/mg catalyst, more preferably at least 30 μm·min/mg catalyst, even more preferably at least μmoles·min/mg catalyst and even more preferably at least 80 μmoles·min/mg catalyst, such activity being determined at 180° C. Applicants have prepared an Avicel-MIP functionalized with a carboxylic acid group, wherein 25 mg of catalyst completely hydrolyzed 250 mg of glucan in 8 hours at 180° C., an activity of about 100 μmoles·min/mg catalyst.

Glucose may be converted to ethanol by methods known to those of ordinary skill in the art.

Having generally described this invention, a further understanding can be obtained by reference to certain specific examples which are provided herein for purposes of illustration only and are not intended to be limiting unless otherwise specified.

### General Procedure for Preparation of Carboxylic Acid Derived Molecular Imprinted Catalysts (MIP-CA)

Imprinting molecule (8 g) was slurried in deionized water (400 mL) and concentrated hydrochloric acid (40 mL). The reaction mixture was then heated to 70° C. for 30 minutes. Subsequently, tetraethylorthosilicate (TEOS, 14.8 mL) was added and the mixture was stirred at 70° C. for 3 hours. Subsequently, 3-cyanopropyltriethoxysilane (1.2 mL) was added. The mixture was heated for 70° C. for about 20 hours. After 20 hours, the mixture was moved to an oil bath (100° C.) and aged for 24 hours without stirring. The reaction mixture was cooled and then the gel was separated by centrifuging.

The resulting gel was transferred to a flask and slurried in 50% sulfuric acid (120 mL). The slurry was heated at ˜98° C. for 20 hours. The mixture was cooled and washed with DI water (60 mL×5) then ethanol (60 mL×2). The gel was dried in the vacuum oven for 24 hours (40° C.).

### COMPARATIVE EXAMPLES

### General Procedure for Preparation of Sulfonic Acid Derived Molecular Imprinted Catalysts (MIP)

a. Soluble Imprinting Molecules

Imprinting molecule (8 g) was slurried in deionized water (400 mL) and concentrated hydrochloric acid (40 mL). The reaction mixture was then heated to 70° C. for 30 minutes. Subsequently, tetraethylorthosilicate (TEOS, 14.8 mL) was added and the mixture was stirred at 70° C. for 3 hours. Subsequently, (3-mercaptopropyl)trimethoxysilane (MPTMS, 1.2 mL) and hydrogen peroxide (30% v/v in water, 2.3 mL) were added. The mixture was heated for 70° C. for about 20 hours. After 20 hours, the mixture was moved to oil bath (100° C.) and aged for 24 hours without stirring. Ethanol (150 mL) was added to the reaction flask and the mixture was refluxed for 7 hours to overnight.

The mixture was cooled, centrifuged, and washed with ethanol (50 mL×3). The gel was dried in the vacuum oven for 24 hours (40° C.).

b. Insoluble Imprinting Molecules

Imprinting molecule (8 g) was slurried in deionized water (400 mL) and concentrated hydrochloric acid (40 mL). The reaction mixture was then heated to 70° C. for 30 minutes. Subsequently, tetraethylorthosilicate (TEOS, 14.8 mL) was added and the mixture was stirred at 70° C. for 3 hours. Subsequently, (3-mercaptopropyl)trimethoxysilane (MPTMS, 1.2 mL) and hydrogen peroxide (30% v/v in water, 2.3 mL) were added. The mixture was heated for 70° C. for about 20 hours. After 20 hours, the mixture was moved to an oil bath (100° C.) and aged for 24 hours without stirring. The reaction mixture was cooled and then the gel was separated by centrifuging.

The resulting gel was transferred to a flask and slurried in 50% sulfuric acid (120 mL). The slurry was heated at ˜98° C. for 20 hours. The mixture was cooled and washed with DI water (60 mL×5) then ethanol (60 mL×2). The gel was dried in the vacuum oven for 24 hours (40° C.).

Using an HPLC, size-exclusion column coupled with an electronic light scattering detection method, glucans with degrees of polymerization from 1-7 can be detected. Larger glucans pass right through the column with little or no retention time. In the figures the retention times correspond to glucose (DP1; 33.2 minutes) on the right, to DP7 (14.6 minutes) on the left. The ordinate is the relative percent of the total areas under the curves for that sample. The abbreviations are MIP=molecularly imprinted catalyst; HEC-MIP=imprinted with hydroxyethylcellulose; Avi-MIP=imprinted with Avicel; CA=carboxylic acid. No suffix means sulfonic acid. N-MIP=non-imprinted catalyst.

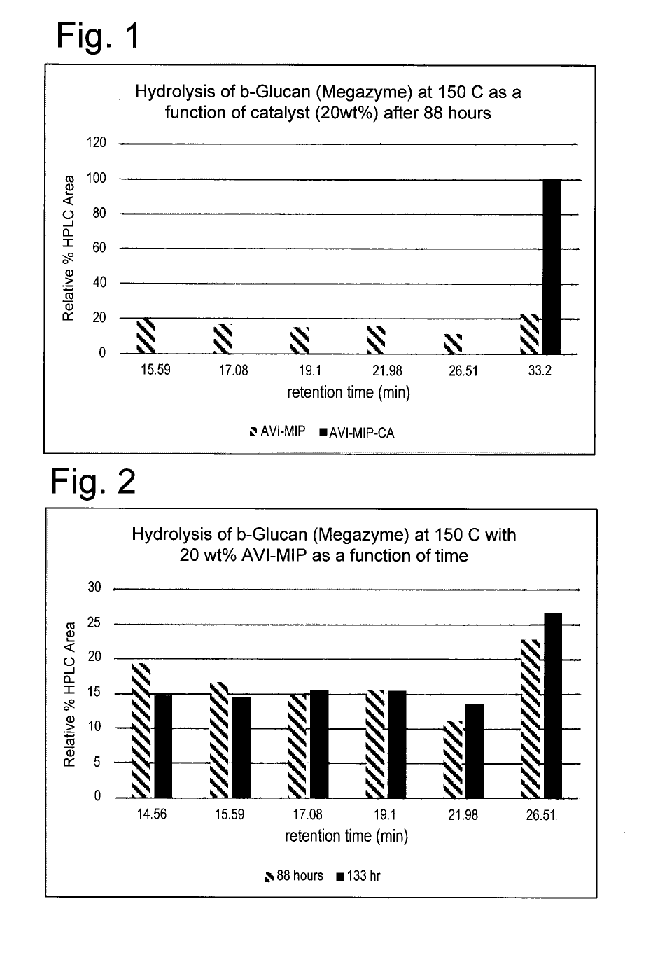

In FIG. 1, hydrolysis of β-glucan by different catalysts at different times is depicted. The superiority of the imprinted catalyst with carboxylic acids is clear.

The hydrolysis of cellobiose with 10 wt % AVI-MIP was also tested. After 138.5 hours, less than 50% of the cellobiose was hydrolyzed. Previously, an experiment with 20 wt % AVI-MIP-CA had almost completely hydrolyzed cellobiose in just 22.5 hours.

The two tubes with Avicel as substrate showed no sign of hydrolysis by HPLC under the same time frame. This may be largely due to a low solubility of the substrate.

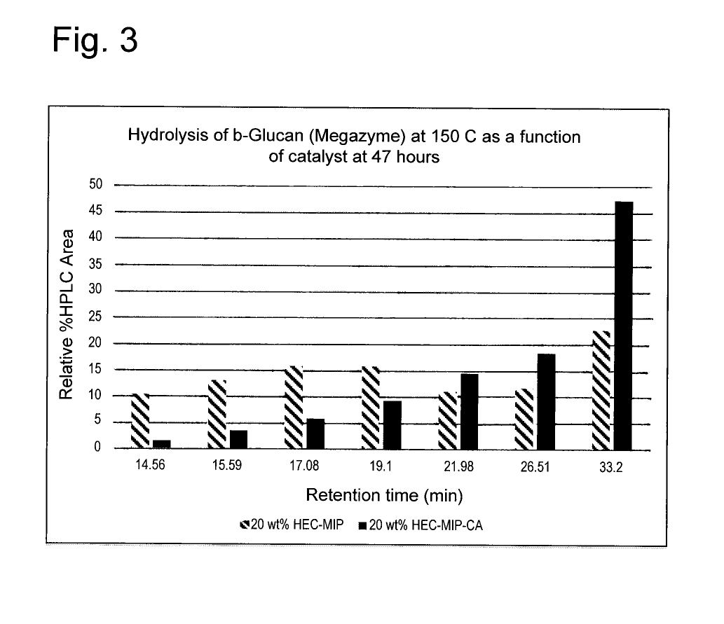

Hydrolysis of β-Glucan (Megazyme) at 150 C was also run using 20 wt % HEC-MIP and 20 wt % HEC-MIP-CA. As with the Avicel imprinted catalysts, the carboxylic acid derived MIP was faster than the sulfonic acid derived catalyst. HPLC data for the reaction with HEC-MIP also still showed significant material with fragments greater than 7 unit whereas most of the starting β-glucan appears consumed with 20 wt % HEC-MIP. These data are shown in FIG. 3.

Obviously, numerous modifications and variations of the present invention are possible in light of the above teachings. It is therefore to be understood that within the scope of the appended claims, the invention may be practiced otherwise than as specifically described herein.

What is claimed as new and desired to be secured by Letters Patent of the United States is:

1. A biomimetic catalyst, said catalyst comprising:
a polymeric silica matrix,
at least one active site imprinted into said matrix, and
at least one carboxylic acid functionality in said active site.

2. The biomimetic catalyst of claim 1, wherein said catalyst is capable of catalyzing hydrolysis of at least one glucose substrate.

3. The bimimetic catalyst according to claim 1 wherein said catalyst has an activity of at least 20 μmoles·min/mg catalyst.

4. The bimimetic catalyst according to claim 1 wherein said catalyst has an activity of at least 80 μmoles·min/mg catalyst.

5. The biomimetic catalyst of claim 1, prepared by reacting at least one tetraorthosilicate and at least one functionalized silane in the presence of at least one imprinting molecule to form a polymeric silica matrix impregnated with said at least one imprinting molecule, wherein at least 1 mol percent of said at least one functionalized silane comprises a carboxylic acid or carboxylic acid precursor functionalized silane; isolating said impregnated polymeric silica matrix; and removing said at least one imprinting molecule from said impregnated polymeric silica matrix via washing or burning to form a silica matrix imprinted with the structure of said at least one imprinting molecule.

6. The biomimetic catalyst of claim 1, wherein said at least one functionalized silane comprises a carboxylic acid precursor and said process further comprises hydrolyzing said carboxylic acid precursor.

7. The biomimetic catalyst of claim 6, wherein said carboxylic acid precursor is a nitrile group.

8. The biomimetic catalyst of claim 1, wherein said at least one imprinting molecule is selected from the group consisting of cellobiose, hydroxyethyl cellulose, Avicel, cellulose and beta glucan.

9. The biomimetic catalyst of claim 1, wherein at least two imprinting molecules are used.

10. A method of preparing a biomimetic catalyst, said method comprising: reacting at least one tetraorthosilicate and at least one functionalized silane in the presence of an imprinting molecule to form a polymeric silica matrix impregnated with said imprinting molecule, wherein at least 1 mol percent of said at least one functionalized silane comprises a carboxylic acid or carboxylic acid precursor functionalized silane; isolating said impregnated polymeric silica matrix; and removing said imprinting molecule from said impregnated polymeric silica matrix via washing or burning to form a silica matrix imprinted with the structure of said imprinting molecule.

11. The method of claim 10, wherein said at least one functionalized silane comprises a carboxylic acid precursor and said process further comprises hydrolyzing said carboxylic acid precursor.

12. A method of producing glucose comprising: dissolving a glucose substrate in a solvent; contacting said glucose substrate in said solvent with the biomimetic catalyst of claim 1 to hydrolyze said glucose substrate to glucose.

13. A method of producing ethanol comprising: dissolving a glucose substrate in a solvent; contacting said glucose substrate in said solvent with the biomimetic catalyst of claim 1 to hydrolyze said glucose substrate to glucose; isolating said glucose; and converting said glucose to ethanol.

14. A method of processing a cellulosic biomass comprising: dissolving a cellulosic biomass material in a solvent; contacting said cellulosic biomass in said solvent with the biomimetic catalyst of claim 1 to hydrolyze said cellulosic biomass to glucose.
